(19) United States
(12) Patent Application Publication
(10) Pub. No.: US 2007/0118926 A1
(43) Pub. Date: May 24, 2007

(54) Engineering broad spectrum virus disease resistance in plants based on the regulation of expression of the RNA dependant RNA polymerase 6 gene

(76) Inventors: Thomas Jack Morris, Lincoln, NE (US); Feng Qu, Lincoln, NE (US)

Correspondence Address: MCKEE, VOORHEES & SEASE, P.L.C., 801 GRAND AVENUE, SUITE 3200, DES MOINES, IA, 50309-2721, US

(21) Appl. No.: 11/602,084

(22) Filed: Nov. 20, 2006

Related U.S. Application Data
(60) Provisional application No. 60/738,262, filed on Nov. 18, 2005.

(51) Int. Cl. A01H 5/00; C07H 21/04; C12N 5/04; A01H 1/00; C12N 15/82

(52) U.S. Cl. 800/280; 800/320.1; 800/312; 435/468; 435/412; 435/415; 536/23.6

(57) ABSTRACT

The present invention provides a novel plant engineered to have a broad spectrum of resistance to plant virus infection by transforming the plant with a polynucleotide construct having an RNA dependant RNA polymerase 6 operably linked to a promoter sequence to allow expression of RNA dependant RNA polymerase 6 in the plant. Also disclosed is a method for conferring on a plant resistance to a broad spectrum of plant virus infection by transforming the plant with a polynucleotide construct having an RNA dependant RNA polymerase 6 operably linked to a promoter sequence.

### CROSS-REFERENCE TO RELATED APPLICATIONS

This application claims priority under 35 U.S.C. § 119 to U.S. Provisional Patent Application No. 60/738,262, filed Nov. 18, 2005, herein incorporated by reference in its entirety.

### FIELD OF THE INVENTION

The present invention relates to novel plants with broad spectrum resistance to plant virus infection. The present invention also relates to novel constructs and methods for transforming plants with broad spectrum resistance to plant viruses.

### BACKGROUND OF THE INVENTION

RNA silencing is a surveillance and defense mechanism occurring in eukaryotic organisms. It is believed to function primarily in defending eukaryotic cells against RNA molecular parasites, such as RNA viruses and transposon RNAs. RNA silencing is triggered by double-stranded RNA (dsRNA) that is subsequently digested by a dsRNA-specific RNase into a small RNA species of 21 to 25 nucleotides long, called small interfering RNA (siRNA). The resultant siRNAs are then recruited into the RNA-induced silencing complex to direct the degradation of other RNAs with sequence complementary to siRNAs. The term RNA silencing generally refers to the suppression of expression of a gene. The extent to which the gene expression is suppressed may vary from partial silencing of the gene to elimination of expression of the gene.

The plant RNA silencing pathway can be divided into two stages, initiation and maintenance. The initiation stage is characterized by its dependence on the trigger dsRNA and siRNAs directly derived from the trigger. The maintenance stage is independent of the trigger and is responsible for the persistent silencing, even after the inducer dsRNA is cleared from the cells. At this stage, RNA silencing is maintained through secondary synthesis of dsRNA by a cellular RNA-dependent RNA polymerase (RdRP), using the siRNA-complementary target RNA as a template.

In addition to guarding the host against parasitic RNAs, recent studies have shown that processes highly related to RNA silencing are also involved in developmental regulation, methylation of chromosomal DNA and histones, and chromatin maintenance. miRNA-mediated regulation of gene expression in both animal and plant systems is a particularly interesting discovery. Unlike siRNAs, miRNAs are encoded by genomes of eukaryotes in the form of partially double-stranded precursor molecules, which are processed by Dicer-like RNase(s) to release mature miRNAs. The miRNAs then mediate degradation or translational repression of the target RNAs. One well-studied example in plants is miR165/166. This miRNA targets the mRNA of three class III homeodomain leucine zipper (HD-ZIP III) transcription factors, PHABULOSA (PHB), PHAVOLUTA (PHV), and REVOLUTA (REV), for cleavage. Restricted expression of these genes in the shoot apex plays a critical role in patterning adaxial-abaxial polarity in lateral organs. Gain-of-function mutations of PHB, PHV, and REV, which cause adaxialization of leaves and vascular systems, have been mapped mostly to the target sites of miR165/166 and have been found to prevent the miRNA-mediated degradation of their mRNAs. Notably, similar phenotypes have also been frequently observed with transgenic plants expressing virus-encoded silencing suppressors and occasionally with virus-infected plants, supporting the argument that siRNA- and miRNA-mediated pathways are closely related.

SDEI/SGS2/RDR6 (RDR6) is an RNA-dependent RNA polymerase (RdRP) from Arabidopsis. RDR6 was previously known to have a critical role in maintaining the posttranscriptional silencing of transgenes in Arabidopsis. RDR6 has been shown to be necessary for the continued silencing of a transgene after the complete elimination of inducer RNA, the cell-to-cell movement of the RNA silencing signal, and the spread of silencing along the target RNA to sequences beyond the region that is homologous to the trigger molecule. The present inventor has demonstrated that RDR6 is most likely responsible for limiting invasiveness by plant viruses into plant tissues. This based on data showing that plants engineered to have a decreased or non-existent level of RDR6 expression showed an increase in viral invasiveness. This discovery demonstrates that RDR6 is actively involved in defending both differentiated and apical plant tissues from invasion by several different RNA plant viruses, including members of the genera Potexvirus, Carmovirus, and Tobamovirus. The consequence of RDR6 down-regulation may depend on both the plant growth temperature and the nature of the invading virus, reflecting the balance between the efficacy of the host RNA silencing and the ability of the invading virus to counteract this process.

It would be desirable to engineer plants to have broad spectrum resistance to viral invasion. In one aspect of the present invention, a novel means for creating plants with increased resistance to a broad spectrum of plant viruses is provided by transforming plants with a modified RDR6 gene under the regulation of a constitutive promoter such as the cauliflower mosaic 35S promoter. This would result in over-expression of the RDR6 gene and hence confer increased resistance in the plant to viral invasiveness.

### BRIEF SUMMARY OF THE INVENTION

In one embodiment of the present invention, a polynucleotide construct including an RNA dependant RNA polymerase 6 operably linked to a promoter sequence to allow the expression of the RNA dependant RNA polymerase 6 in plants is provided. The promoter may be, but is not limited to, a constitutive promoter, an inducible promoter, or a tissue preferred promoter.

In another embodiment of the present invention, a plant transformed with a polynucleotide construct including an RNA dependant RNA polymerase 6 operably linked to a promoter sequence to allow expression of RNA dependant RNA polymerase 6 in plants is provided. The plant may be selected from the group of cereals, pulses, tubers and seed crops.

In yet another embodiment of the present invention, an expression vector including a construct having an RNA dependant RNA polymerase 6 operably linked to a promoter sequence to allow expression of RNA dependant RNA polymerase 6 in plants is provided.

In yet another embodiment of the present invention, a plant host cell comprising a construct comprising an RNA dependant RNA polymerase 6 operably linked to a promoter sequence to allow expression of RNA dependant RNA polymerase 6 in the plant host cell is provided.

In still yet another embodiment of the present invention, a method of conferring on a plant resistance to a broad spectrum of plant viruses including the step of transforming the plant with a polynucleotide construct having an RNA dependant RNA polymerase 6 operably linked to a promoter sequence to allow expression of RNA dependant RNA polymerase 6 in the plant is provided. The plant viruses which the plant is conferred resistance to may include, but are not limited to, viruses from the genera Potexvirus, Carmovirus, and Tobamovirus.

It is an object of the present invention to provide a plant with increased expression of the RNA dependant RNA polymerase 6 gene.

Another object of the present invention is to provide a plant with broad spectrum resistance to plant virus infection.

Yet another object of the present invention is to provide a plant with resistance to viruses from at least the genera Potexvirus, Carmovirus, and Tobamovirus.

Still another object of the present invention is to provide a construct which includes the RNA dependant RNA polymerase 6 gene and a promoter operably linked to the RNA dependant RNA polymerase 6 gene.

A further object of the present invention is to transform a plant with a construct which includes the RNA dependant RNA polymerase 6 gene and a promoter operably linked to the RNA dependant RNA polymerase 6 gene.

It is an object of the present invention to provide a method for conferring resistance on a plant to a broad spectrum of plant viruses.

Yet another object of the present invention is to reduce crop damage caused by virus infections in many major crop plants.

The means and methods of accomplishing one or more of these and/or other objectives will become apparent from the detailed description of the invention and the description of the drawings, which follows hereafter.

### BRIEF DESCRIPTION OF THE DRAWINGS

FIG. 1 shows potato virus X (PVX)-mediated virus induced gene silencing of NbRDR6 disrupts silencing of a GFP transgene in N. benthamiana 16c plants. The section of FIG. 1 labeled A shows images of systemically silenced 16c plants infected with the PVX vector (top) and PVX-NbRDR6 (bottom), showing that the silenced status of GFP was disrupted by VIGS of NbRDR6. The section of FIG. 1 labeled B shows total RNA extracted from plants infected with either the PVX vector alone or the vector containing the sequence of either NbRDR1 or NbRDR6 were subjected to RNA blot hybridization using the probes indicated to the right of the panels. The bottom panel shows the ethidium bromide-stained gel, serving as the loading control.

FIG. 2 shows transgenic dsRDR6 N. benthamiana plants are more susceptible to turnip crinkle virus (TCV) infection at higher growth temperature. The section of FIG. 2 labeled A shows wild-type and dsRDR6 plants infected with TCV at 21 and 27° C. at 14 dpi. The dsRDR6 plants kept at 27° C. show significantly greater symptom severity and stunting. The section of FIG. 2 labeled B shows total RNA samples extracted from both wild-type and dsRDR6 plants, mock infected or TCV infected, reared at 21 or 27° C., were subjected to RNA blot hybridization with the probes indicated to the right of the panels. Hybridization with the TCV probe (top) was performed using 0.5 μg of total RNA from each sample, showing a marked reduction of TCV RNA accumulation in the 27° C. WT samples. In the second and third panels, RNA extracts of 5 μg each were hybridized with NbRDR6 and NbRDR1 probes, respectively. The NbRDR6 mRNA was consistently undetectable in dsRDR6 plants (panel 2). The lower level of NbRDR6 mRNA seen in lanes 5 and 6 likely reflects the reduced proportion of cellular RNA due to the very high levels of TCV RNA in the infected samples (panel 2). The NbRDR1 mRNA levels are highly variable and do not correlate with that of NbRDR6 mRNA. The bottom panel is an ethidium bromide-stained gel serving as the loading control gRNA genomic RNA.

FIG. 3 shows transgenic dsRDR6 plants permit higher levels of PVX replication than do wild-type plants at both temperatures tested. The section of FIG. 3 labeled A shows PVX-infected plants at 14 dpi showing slightly greater symptom severity in the dsRDR6 plants kept at 27° C. The section of FIG. labeled B shows RNA blot hybridization using a PVX-specific probe, showing that PVX viral RNA accumulated to higher levels at both 21 and 27° C. in the dsRDR6 plants than in the wild-type plants.

FIG. 4 shows transgenic dsRDR6 plants infected with green fluorescent protein (GFP)-tagged strain of tobacco mosaic virus (TMV) (TMV-GFP) show greater stunting than wild-type plants at 27° C. The section of FIG. 4 labeled A shows TMV-GFP-infected plants at 21 dpi. The section of FIG. 2 labeled B shows RNA blot hybridization using a TMV-specific probe, showing that viral RNA accumulation levels were similar for wild-type and dsRDR6 plants at both temperatures.

FIG. 5 shows TMV-GFP efficiently colonizes the shoot apices of transgenic dsRDR6 plants, causing developmental defects in leaves and flowers. The section of FIG. 5 labeled A shows in panel 1 TMV-GFP-infected wild-type (left) and dsRDR6 (right) plants kept at 21° C. for 5 weeks. The leaves on the dsRDR6 plants have a distinctive crab-leg-like appearance. Panel 2 shows characteristic deformations seen with selected leaves of the TMV-GFP-infected dsRDR6 plants kept at 21° C. Panels 3 and 4 show similar leaf abnormalities seen with TMV-GFP-infected dsRDR6 plants kept at 27° C. Panel 5 shows typical flower abnormality observed with the TMV-GFP-infected dsRDR6 plants kept at 27° C. Panel 6 shows flower from a noninfected dsRDR6 plant kept at 27° C. The section of FIG. 5 labeled B shows in panel I total RNA was extracted from the apical tissues of the plants treated as described in Results and subjected to RNA blot hybridization with TMV, and in panel II, NbPHV, and in panel III, actin probes. The numbers underneath panel II show the relative levels of NbPHV mRNAs, determined by densitometry (Molecular Dynamics). The same RNA samples were also subjected to hybridization to illustrate the level of miR165 (panel IV). The section of FIG. 5 labeled C shows confocal microscopic images of plant shoot apices. Microscopic parameters, including laser settings, were the same for all images in the six panels. Bar=100 μm. Each panel shows two different images, with the left image reflecting the GFP signal and the right image showing the result of merging the GFP signal with that of the chlorophyll autofluorescence which depicts the organ shape. Panels 1 and 2 show mock-inoculated dsRDR6 plants. Panels 3 and 4 show TMV-GFP-infected WT plants. Panels 5 and 6 show TMV-GFP-infected dsRDR6 plants. Panels 1, 3, and 5 each depict an F1 floral primordium. Panels 2, 4, and 6 depict a more developed but unopened flower (F3), with the flower in panel 6 being manually opened. Arrows highlight GFP fluorescence.

### DETAILED DESCRIPTION

The present inventor has determined a novel means for engineering plants with broad spectrum resistance to plant virus infection. The plant gene RDR6 is responsible for decreased invasiveness by plant viruses into plant tissues and is a critical component of the RNA silencing-based antiviral defense operating in the plant shoot apices as well as differentiated leaf tissue. The role of the RDR6-mediated RNA silencing pathway is a form of general antiviral defense directed against a broad spectrum of viruses. Through the use of GFP-tagged tobacco mosaic virus, RDR6 has been shown to play a critical role in defending shoot apices from virus invasion in plants. By incapacitating RDR6, rigorous replication of GFP-tobacco mosaic virus was enabled in flower meristems located in plant shoot tips. RDR6 also was demonstrated to have an important role in the antiviral defense in differentiated leaf tissue. Transgenic plants greatly diminished in RDR6 expression were used to test various RNA viruses belonging to distinct virus families for changes in susceptibility. Each virus displayed a definite increase in invasiveness that was affected by the temperature at which the plant was grown.

Viral invasiveness and hence the outcome of a viral infection is critically influenced by the balance between the robustness of RNA silencing-based defense and the relative strength of the viral silencing suppressor. Viruses with stronger silencing suppressors are also more likely to overcome RNA silencing-based defense weakened by lower growth temperatures, thus minimizing the impact of RNA silencing disruption on virus infection at these temperatures.

According to one aspect of the present invention there is therefore provided, preferably within a vector suitable for stable transformation of a plant cell, a polynucleotide construct in which a promoter is operably linked for transcription of the RDR6 gene in a plant cell. The constructs used in the experimental exemplification described in this application are based on cDNA of several different viruses, including the potato virus X (PVX); tobacco mosaic virus (TMV); and turnip crinkle virus (TCV). Any RNA or DNA plant virus may be used in generation of constructs in accordance with the present invention in a manner that is similar to that described here for PVX, TMV, and TCV, including but not limited to tobacco etch virus (Dolja, V. V., et al (1992), Proc. Natl. Acad. Sci. USA, 89: 10208-10212), tobacco rattle virus (Ziegler-Graff, V., et al (1991), Virology, 182: 145-155), tomato bushy stunt virus (Scholthof, H. B., et al (1993), Mol. Plant-Microbe Interact., 6: 309-322), brome mosaic virus (Mori, M., et al (1993), J. Gen. Virol., 74: 1255-1260), cauliflower mosaic virus (Futterer, J. and Hohn, T. (1991), EMBO J., 10: 3887-3896), African cassava mosaic virus (Ward, A., et al (1988), EMBO J., 7: 1583-1587), tomato golden mosaic virus.

The polynucleotide construct, preferably comprising a transformation/expression vector, is engineered to incorporate the RDR6 gene. The methodologies used for isolating and cloning the RDR6 gene may include identification of the gene by hybridization with probes, PCR, probe/primer/synthetic gene synthesis, sequencing, molecular cloning and other techniques which are well-known to those skilled in molecular biology.

The function of the promoter in the construct is to ensure that the polynucleotide sequences are transcribed. By “promoter” it is meant a sequence of nucleotides from which transcription may be initiated. “Operably linked” means that the promoter is suitably positioned and oriented for transcription to be initiated from the promoter.

In one aspect of the invention the preferred promoters includes the constitutive cauliflower mosaic 35S promoter. The cauliflower mosaic 35S promoter is expressed in many, if not all, cell types of many plants. (Sanders, P. R., et al (1987), Nucleic Acids Res., 15: 1543-1558). Other constitutively expressed promoters, such as the nopaline synthase promoter of Agrobacterium tumefaciens, may be used effectively as components of the construct comprising the RDR6 gene.

In another aspect of the invention, other promoters including those that are inducible or tissue preferred may be used. For example, in one aspect of the invention a construct could be engineered so that the RDR6 gene is expressed under the regulation of a promoter of plant origin whose expression is highly induced by virus infections. Plants comprising the construct engineered with RDR6 regulated in this fashion would then express RDR6 when attacked by the virus, and accordingly confer a strong resistance response in the plant. Inducible promoters may be advantageous in certain circumstances because they place the timing of reduction in expression of the target gene of interest under the control of the user.

Expression under the control of an inducible promoter is “switched on” or increased in response to an applied stimulus. The nature of the stimulus varies between promoters. Some inducible promoters cause little or undetectable levels of expression (or no expression) in the absence of the appropriate stimulus. Other inducible promoters cause detectable constitutive expression in the absence of the stimulus. Preferably, the level of expression increases upon application of the relevant stimulus.

In another aspect of the invention, a construct could be engineered so that expression of the RDR6 gene is driven by a seed specific promoter, such as the soybean promoter of β-conglycinin, also known as the 7S protein, which drives seed-directed transcription, Bray, Planta 172: 364-370 (1987); and seed-directed promoters from the zein genes of maize endosperm, Pedersen et al., Cell 29: 1015-26 (1982). Promoters that are both tissue specific and inducible by specific stimuli may also be used.

In one aspect of the present invention, a typical polynucleotide construct, preferably comprising a transformation/expression vector, may contain some or all of the following elements: a cloning site for insertion of an exogenous polynucleotide sequence, which would code for RDR6; eukaryotic polynucleotide elements that control initiation of transcription of the exogenous gene, such as a promoter; and polynucleotide elements that control the processing of transcripts, such as a transcription termination/polyadenylation sequence. In another aspect of the invention, the vector also can contain such sequences as are needed for the eventual integration of the vector into the chromosome of the transformed plant.

In an additional embodiment of the present invention, the polynucleotide construct comprising a transformation/expression vector may be used in transformation of one or more plant cells to introduce the construct stably into the genome, so that it is stably inherited from one generation to the next. This is preferably followed by regeneration of a plant from such cells to produce a transgenic plant. Thus, in further aspects, the present invention also provides the use of the construct or vector in production of a transgenic plant, methods of transformation of cells and plants, plant and microbial (particularly Agrobacterium) cells, and various plant products.

For introduction into a plant cell, the nucleic acid construct may be in the form of a recombinant vector, for example an Agrobacterium binary vector. Microbial, particularly bacterial and especially Agrobacterium, host cells containing a construct according to the invention or a vector which includes such a construct, particularly a binary vector suitable for stable transformation of a plant cell, are also provided by the present invention.

Successfully transformed cells and/or plants may be selected following introduction of the nucleic acid into plant cells, optionally followed by regeneration into a plant, for example by using one or more marker genes such as antibiotic resistance. Selectable genetic markers may be used consisting of chimaeric genes that confer selectable phenotypes such as resistance to antibiotics such as kanamycin, hygromycin, phosphinotricin, chlorsulfuron, methotrexate, gentamycin, spectinomycin, imidazolinones and glyphosate.

Plants transformed with the DNA segment containing the sequence may be produced by standard techniques which are already known for the genetic manipulation of plants. DNA can be transformed into plant cells using any suitable technology, such as a disarmed Ti-plasmid vector carried by Agrobacterium exploiting its natural gene transfer ability (EP-A-270355, EP-A-0116718, NAR 12(22) 8711-87215 1984), particle or microprojectile bombardment (U.S. Pat. No. 5,100,792, EP-A-444882, EP-A-434616) microinjection (WO 92/09696, WO 94/00583, EP 331083, EP 175966, Green et al. (1987) Plant Tissue and Cell Culture, Academic Press), electroporation (EP 290395, WO 8706614) other forms of direct DNA uptake (DE 4005152, WO 9012096, U.S. Pat. No. 4,684,611), liposome mediated DNA uptake (e.g. Freeman et al. Plant Cell Physiol. 29: 1353 (1984)), or the vortexing method (e.g. Kindle, PNAS U.S.A. 87: 1228 (1990d). Physical methods for the transformation of plant cells are reviewed in Oard, 1991, Biotech. Adv. 9: 1-11.

Agrobacterium transformation is widely used by those skilled in the art to transform dicotyledonous species. Recently, there has been substantial progress towards the routine production of stable, fertile transgenic plants in almost all economically relevant monocot plants (Toriyama, et al. (1988) Bio/Technology 6, 1072-1074; Zhang, et al. (1988) Plant Cell Rep. 7, 379-384; Zhang, et al. (1988) Theor Appl Genet 76, 835-840; Shimamoto, et al. (1989) Nature 338, 274-276; Datta, et al. (1990) Bio/Technology 8, 736-740; Christou, et al. (1991) Bio/Technology 9, 957-962; Peng, et al. (1991) International Rice Research Institute, Manila, Philippines 563-574; Cao, et al. (1992) Plant Cell Rep. 11, 585-591; Li, et al. (1993) Plant Cell Rep. 12, 250-255; Rathore, et al. (1993) Plant Molecular Biology 21, 871-884; Fromm, et al. (1990) Bio/Technology 8, 833-839; Gordon-Kamm, et al. (1990) Plant Cell 2, 603-618; D'Halluin, et al. (1992) Plant Cell 4, 1495-1505; Walters, et al. (1992) Plant Molecular Biology 18, 189-200; Koziel, et al. (1993) Biotechnology 11, 194-200; Vasil, I. K. (1994) Plant Molecular Biology 25, 925-937; Weeks, et al. (1993) Plant Physiology 102, 1077-1084; Somers, et al. (1992) Bio/Technology 10, 1589-1594; WO92/14828). In particular, Agrobacterium mediated transformation is now emerging also as a highly efficient transformation method in monocots (Hiei et al. (1994) The Plant Journal 6, 271-282).

The generation of fertile transgenic plants has been achieved in the cereals rice, maize, wheat, oat, and barley (reviewed in Shimamoto, K. (1994) Current Opinion in Biotechnology 5, 158-162; Vasil, et al. (1992) Bio/Technology 10, 667-674; Vain et al., 1995, Biotechnology Advances 13 (4): 653-671; Vasil, 1996, Nature Biotechnology 14 page 702).

Microprojectile bombardment, electroporation and direct DNA uptake are preferred where Agrobacterium is inefficient or ineffective. Alternatively, a combination of different techniques may be employed to enhance the efficiency of the transformation process, for example bombardment with Agrobacterium coated microparticles (EP-A-486234) or microprojectile bombardment to induce wounding followed by co-cultivation with Agrobacterium (EP-A-486233).

Following transformation, a plant may be regenerated, e.g. from single cells, callus tissue or leaf discs, as is standard in the art. Almost any plant can be entirely regenerated from cells, tissues and organs of the plant. Available techniques are reviewed in Vasil et al., Cell Culture and Somatic Cell Genetics of Plants, Vol. I, II and III, Laboratory Procedures and Their Applications, Academic Press, 1984, and Weissbach and Weissbach, Methods for Plant Molecular Biology, Academic Press, 1989.

Also according to the invention there is provided a plant cell having incorporated into its genome a DNA construct as disclosed. A further aspect of the present invention provides a method of making such a plant cell involving introduction of a vector including the construct into a plant cell. Such introduction should be followed by recombination between the vector and the plant cell genome to introduce the sequence of nucleotides into the genome. RNA encoded by the introduced nucleic acid construct may then be transcribed in the cell and descendants thereof, including cells in plants regenerated from transformed material. A gene stably incorporated into the genome of a plant is passed from generation to generation to descendants of the plant, so such descendants should show the desired phenotype. The present invention also provides a plant comprising a plant cell as disclosed.

The present invention is not limited to a certain variety of plants. Without limitation, the present invention can be used in crop plants, including cereals and pulses, maize, wheat, potatoes, tapioca, rice, sorghum, millet, cassava, barley, pea and other root, tuber or seed crops. Important seed crops for which the invention may be used include, but are not limited to, oil seed rape, sugar beet, maize, sunflower, soybean and sorghum.

The present invention is illustrated by the following examples. The foregoing and following description of the present invention and the various embodiments are not intended to be limiting of the invention but rather are illustrative thereof. Hence, it will be understood that the invention is not limited to the specific details of these examples.

### EXAMPLES

### Example 1

### Cloning of Full-Length cDNA of NbRDR6

The tomato-expressed sequence tag EST360431 was identified by BLAST searches of The Institute of Genome Research (TIGR) database as being most closely related to Arabidopsis RDR6. Because the corresponding genes of tomato and N. benthamiana share high sequence homology at the nucleotide level, the sequence of EST360431 was used to design primers for successfully amplifying a cDNA fragment from N. benthamiana by reverse transcription coupled with PCR. The sequence of the amplified fragment served as the basis for further efforts in obtaining full-length cDNA of NbRDR6 by use of the procedure of rapid amplification of cDNA ends.

### Example 2

### Virus-Induced Gene Silencing (VIGS) or NbRDR6

A modified potato virus X (PVX) vector was used and the EcoRV and Not1 cloning sites were used to insert a 1,112-bp NbRDR6 fragment (nt 167 to 1288 of its cDNA) to produce PVX-NbRDR6. Infectious transcripts of the construct were used to infect N. benthamiana plant leaves, which were collected at 5 days post inoculation (dpi) and used for further infection.

### Example 3

### Virus Stocks and Inoculations

Turnip crinkle virus (TCV) was propagated from infectious transcripts of pT1d1. Capped transcripts of the PVX vector were used as the inocultim to propagate PVX. Tobacco mosaic virus (TMV) inoculum was propagated from infectious transcripts of the cDNA clone of a green fluorescent protein (GFP)-tagged strain of TMV (TMV-GFP). Groups containing at least four infected plants each were reared under the conditions described in Examples 6 and 7, and the experiments were repeated at least three times.

### Example 4

### Generation of Transgenic N. benthamiana Plants Expressing dsRNA Targeting NbRDR6

Two fragments of NbRDR6 cDNA, 630 by (nt 2970 to 3600) and 1,133 by (nt 3811 to 2678) in length, were cloned into the vector pRTL2 (1) downstream of the cauliflower mosaic virus 35S promoter, with the 630-bp fragment in the sense orientation and the 1,133-bp fragment in the antisense orientation. The larger fragment contained the whole sequence of the smaller one, so that transcription in plants would produce NbRDR6-specific dsRNA. The complete cassette, including the 35S promoter and terminator sandwiching the insert, was then subcloned to the binary vector pPZP212. The resulting construct, pPZP-dsRDR6, was brought into Agrobacterium sp. strain C58C1, which was used to transform N. benthamiana leaf disks.

### Example 5

### RNA Blot Hybridizations

N. benthamiana plants with five to six true leaves were inoculated on the first two true leaves, and the infected plants were subject to RNA extraction 2 to 3 weeks after infection. To ensure the data generated between different treatments are comparable, the first systemic leaf was counted as the topmost emerging leaf with the length of the main vein being at least 1 cm and, except for some rare cases specifically noted in Example 10, picked the third systemic leaves of the infected plants for RNA extraction. To detect mRNA of NbRDRI (a previously identified RdRP from N. benthamiana), NbRDR6, NbPHV (the N. benthamiana homolog of PHV), and NbACT (N. benthamiana actin), 5 ftg of total RNA of each sample was subjected to RNA blot hybridization with in vitro-transcribed RNA probes of approximately 800 nt long. The NbPHV-specific probe was synthesized from an 800-bp fragment of NbPHV cDNA containing a T7 promoter at its 5′ end. This sequence is highly homologous to that of Nicotiana sylvestris PHV. The NbACT probe was generated by reverse transcription-PCR using primers based on the tobacco actin sequence. The hybridizations were carried out at 68° C. in UltraHyb buffer (Ambion, Austin, Tex.). For detection of miR165, low-molecular-weight RNA was enriched from total RNA samples and subjected to hybridization with the end-labeled complementary oligonucleotide at 35° C. in UltraHyb-Oligo buffer (Ambion, Austin, Tex.). The membranes were washed twice at 42° C. for 30 min each with a buffer containing 2×SSC (1×SSC is 0.15 M NaC1 plus 0.015 M sodium citrate) and 0.5% sodium dodecyl sulfate before exposure to X-ray films. The uppermost (2-mm) regions of plant apices were detached and examined with an Olympus FluoView 500 confocal laser scanning microsco0pe. The cDNA sequence of NbRDR6 has been deposited into GenBank with accession number AY722008.

### Example 6

### Characterization of NbRDR6

N. benthamiana was chosen as the experimental host to investigate the effect of temperature on the antiviral role of RDR6 because it grows well at a relatively wide range of temperatures (at least between 15 and 33° C.) and it is susceptible to a broad spectrum of well characterized plant viruses. Importantly, the RNA silencing process has been shown to be robust in N. benthamiana by numerous previous studies. The sequence of full-length NbRDR6 cDNA was resolved, revealing its amino acid sequence to be 62% identical and 76% similar to Arabidopsis RDR6 but only 37% identical and 53% similar to NbRDR1. In addition, it is only 34% identical and 52% similar to NbRDR2 (GenBank accession number AY722009). Taken together, these data strongly suggest that NbRDR6 is the homolog of Arabidopsis RDR6.

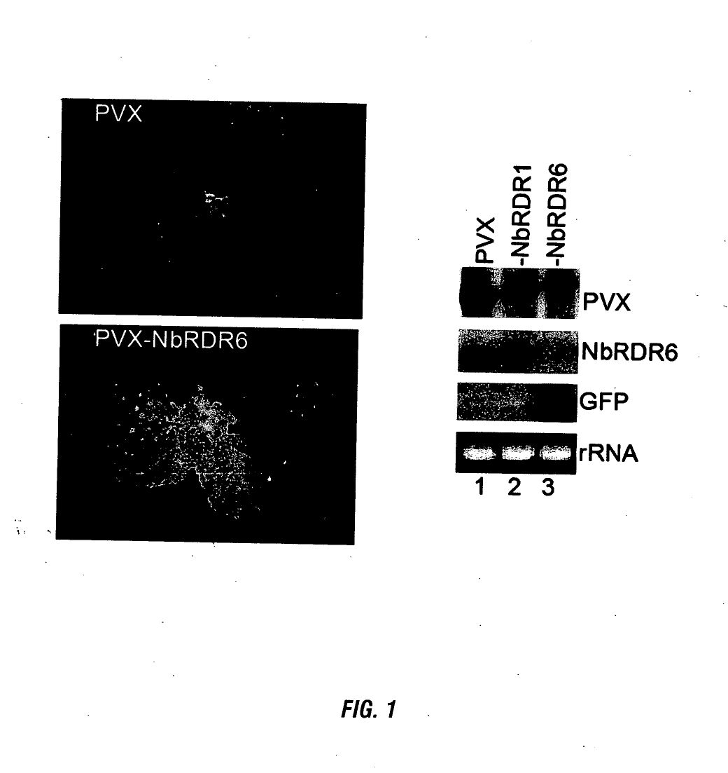

It was next demonstrated, by examining the impact of VIGS-based NbRDR6 down-regulation on transgene silencing, that NbRDR6 also functioned similarly to Arabidopsis RDR6 in RNA silencing maintenance. The GFP 16c line of N. benthamiana plants, which express GFP at high levels but which could be systemically silenced by transiently overexpressing GFP in very young plants, was used. Upon completion of systemic silencing of GFP in the 16c plants, which were monitored using a handheld long-wavelength UV lamp (FIG. 1A), these same plants were infected with PVX-NbRDR6, designed to down-regulate NbRDR6 expression through VIGS. Infection with PVX-NbRDR6 led to reexpression of the silenced GFP transgene (FIG. 1A, bottom), whereas control plants infected with PVX vector alone remained silenced (FIG. 1A, top). As an additional control, infection with a PVX derivative containing a portion of the NbRDR1 sequence (PVX-NbRDR1) also failed to disrupt the silencing of the GFP transgene. RNA blot hybridization with a PVX-specific probe revealed the accumulation levels of genomic RNAs of respective VIGS constructs (FIG. 1A, top). Rehybridization of the same RNA samples with an NbRDR6 probe showed that NbRDR6 mRNA was reduced to below the level of detection by PVX-NbRDR6 infection (FIG. 1B, panel 2, lane 3) but not by either PVX or PVX-NbRDR1 infection (FIG. 1B panel 2, lane 3) but not by either PVX or PVX-NbRDR1 infection (FIG. 1B panel 2, lanes 1 and 2). The reduction of the NbRDR6 mRNA level was accompanied by a corresponding increase of GFP expression (FIG. 1B, panel 3, lane 3).

Previous studies by others have shown that RDR1 plays an important role in antiviral defense with both tobacco and Arabidopsis but that it is nonfinctional in N. benthamiana due to the presence of two premature stop codons in the middle of its mRNA. Here the RNA samples were also hybridized with an NbRDR1 probe. The NbRDR1 mRNA level was reduced by infection with PVX-NbRDR1 but not by infection with PVX-NbRDR6 (FIG. 1B, panel 4), indicating that VIGS of NbRDR6 did not lead to nonspecific targeting of NbRDR1. The NbRDR1 mRNA, similarly to its tobacco counterpart, was detected as two distinct bands (FIG. 1B, panel 4). However, its expression level is rather low even in the absence of VIGS and also highly variable, suggestive of it being an expressed pseudogene. Together, these results verify that NbRDR6 is indeed functionally homologous to Arabidopsis RDR6.

### Example 7

### Transgenic Plants Expressing dsRNA Targeting NbRDR6 Display Enhanced Susceptibility to TCV in a Temperature-Dependent Manner

After the role of NBRDR6 in the maintenance of transgene silencing was established, the same PVX-based VIGS approach was initially used to show that plants down-regulated for NbRDR6 expression were generally more susceptible to subsequent infection by both TCV and TMV. However, the synergy between PVX and the challenger viruses caused very severe necrosis, making molecular verification difficult. It was hence decided to evaluate the role of NbRDR6 in antiviral silencing by using transgenic plants expressing a dsRNA construct targeting NbRDR6. A total of 36 lines of T1 plants were screened for decreased expression of NbRDR6 mRNA with RNA blot hybridization. This screen identified eight lines that showed dramatically lower expression of NbRDR6 mRNA with no visible developmental defects. One of the lines (line 6) was chosen for further experimentation.

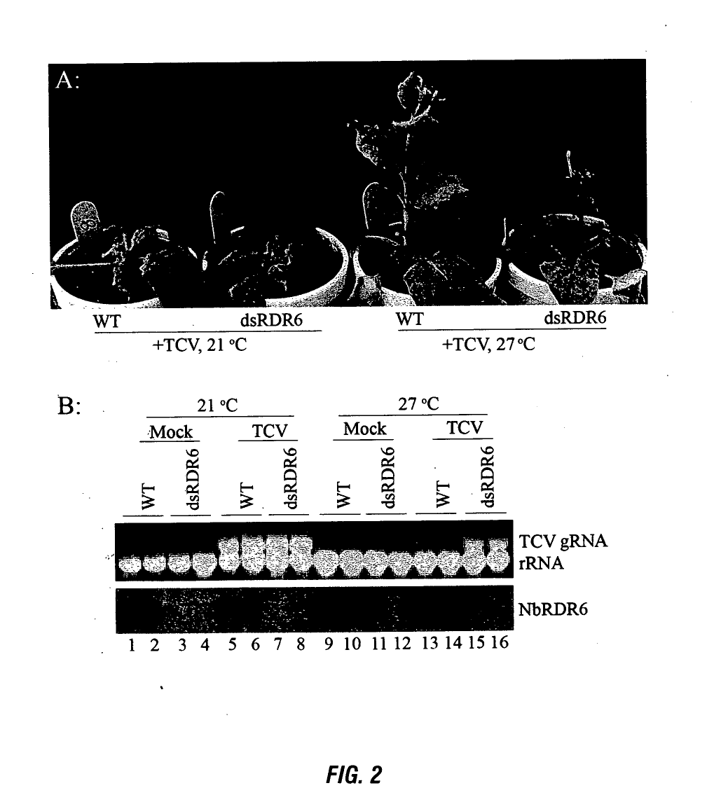

The dsRDR6 plants were infected with TCV and kept the infected plants at 21 and 27° C., respectively, to monitor for possible temperature-dependent effects. The temperature effect on TCV symptoms was evident as early as 7 dpi and was clearly visible at 14 dpi (FIG. 2A). Wild-type (WT) and dsRDR6 plants both showed equally severe symptoms at 21° C., while at 27° C. the dsRDR6 infected plants were evidently more severely diseased and stunted than WT plants. The differences in symptom severity correlated well with levels of viral RNA in the respective plants (FIG. 2B, top). Note that the TCV genomic RNA accumulated to levels nearly equal to that of the 25S rRNA in both WT and dsRDR6 plants at 21° C. (FIG. 2B, bottom, lanes 5 to 8). Importantly, at the higher temperature of 27° C., the level of the viral RNA was dramatically reduced in WT plants (FIG. 2B, top and bottom, compare lanes 5 and 6 to lanes 13 and 14) but less so in dsRDR6 plants (FIG. 2B, compare lanes 7 and 8 to lanes 15 and 16). The RNA samples were also hybridized with an NbRDR6 probe to verify that the NbRDR6 mRNA was consistently below the level of detection in all of the dsRDR6 plants at both temperatures (FIG. 2B, panel 2). These same RNA samples were further subjected to hybridization with an NbRDR1 probe to determine if the dsRNA transgene might also interfere with the expression of other RdRPs. The result (FIG. 2B, panel 3) revealed that the levels of NbRDR1 mRNA, while highly variable, did not correlate with the levels of NbRDR6. The variation in the NbRDR1 mRNA levels could reflect the previous finding that RDR1 in N. benthamiana is likely an expressed pseudogene. In conclusion, these data strongly suggest that NbRDR6 plays a significant role in antiviral defense in N. benthamiana, especially at the higher temperature.

### Example 8

### PVX Viral RNA is More Abundant in dsRDR6 Plants Than in WT Plants at Both Low and High Temperatures

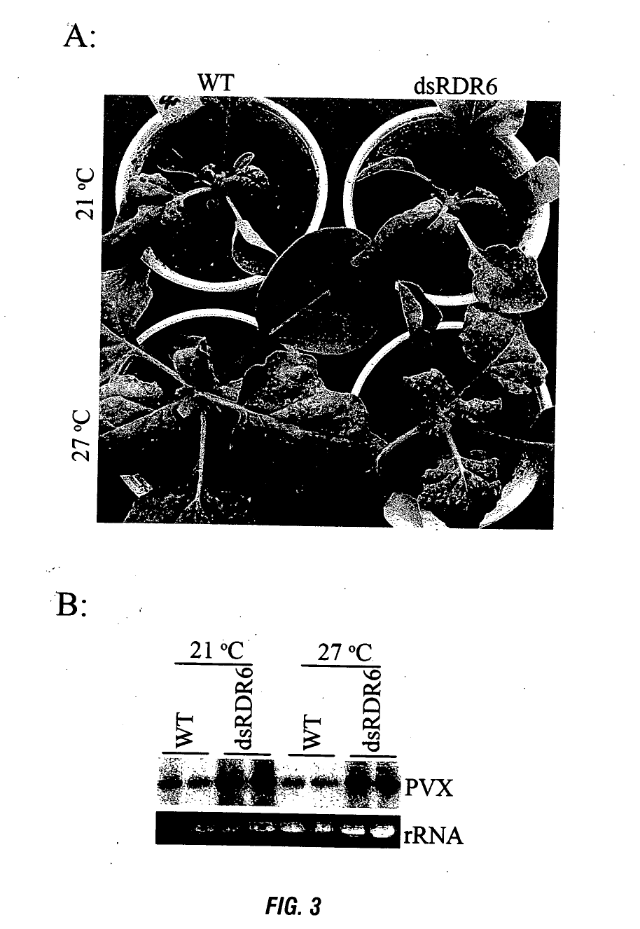

Infection was also tested with a second unrelated RNA plant virus, PVX, on the dsRDR6 plants to determine if the temperature effect was a more general feature of NbRDR6-mediated antiviral defense. The symptoms for PVX at 14 dpi were similar to those observed for TVC. As shown in FIG. 3A, the symptoms of infected dsRDR6 plants were more severe and plant growth was more stunted than for their WT counterparts at the higher incubation temperature (27° C). A significant increase in PVX genomic RNA accumulation was evident with the dsRDR6 plants compared to the WT plants at both temperatures, an indication that the temperature effect was less dramatic for the PVX infections. Again, viral RNA accumulation appeared to be slightly better in both types of plants at 21° C. than at 27° C. (FIG. 3B, top). These results add further support to the conclusion that NbRDR6 plays an important role in antiviral defense. In addition, these results suggest that PVX infection may behave more similarly to cucumber mosaic virus (CMV) infection, in that the impact of NbRDR6 down-regulation was detectable over a broader temperature range.

### Example 9

### TMV-GFP Infection Caused More Severe Plant Stunting in NbRDR6 Down-Regulated Plants Grown at Higher Temperature

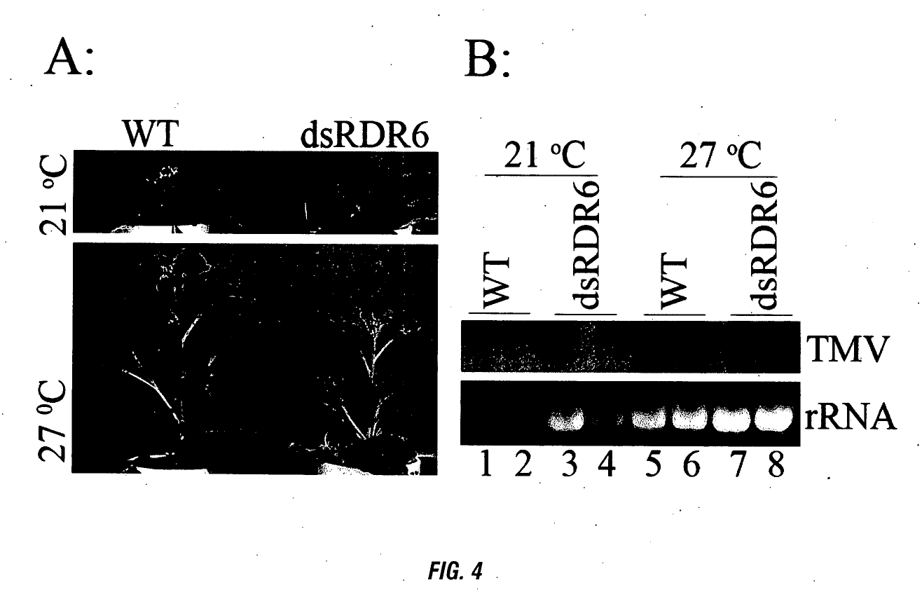

The susceptibility of the dsRDR6 plants to TMV-GFP was tested at the two experimental temperatures so that the effects of temperature could be visually monitored on virus spread in the infected plants. As was observed for the TCV and PVX infections, when kept at 21° C., both WT and dsRDR6 plants infected by TMV-GFP showed comparable symptoms 2 to 3 weeks after infection (FIG. 4A, top). Again, plants kept at 27° C. consistently displayed milder symptoms than their 21° C. counterparts. The differences between infected WT and dsRDR6 plants were more subtle and occurred later than for either the TCV- or the PVX-infected plants (FIG. 4A, bottom). Moreover, it was not consistently possible to correlate the levels of GFP fluorescence and viral RNA accumulation with the degrees of stunting of the infected plants. The difference in degree of stunting likely resulted from the more efficient apical colonization by TMV-GFP in the dsRDR6 plants.

### Example 10

### Down-Regulating the NbRDR6 Expression Promotes TMV Invasion of Shoot Apices

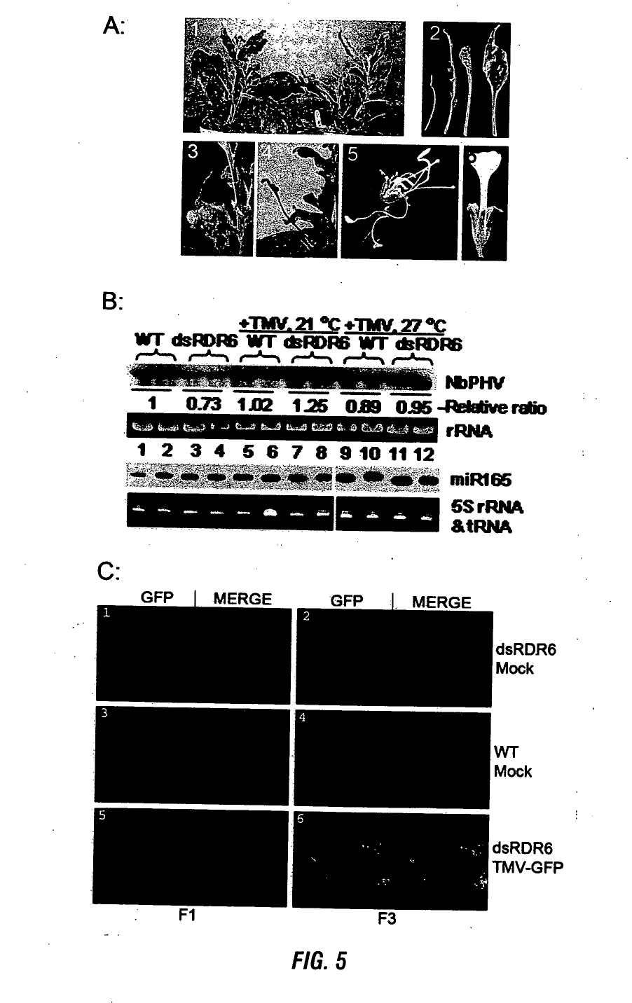

Aside from the obvious stunting of plants held at 27° C., the WT and dsRDR6 plants infected with TMV-GFP displayed very similar symptoms when held for up to 3 weeks after infection. Intriguingly, when the infected plants continued to be monitored for a more extended period of time, highly unusual leaf deformations were observed in infected dsRDR6 plants kept at 21° C., beginning about 5 weeks after infection. As shown in FIG. 5A (panels 1 and 2), the leaves of TMV-GFP-infected dsRDR6 plants kept at 21° C. had odd shapes, ranging from long rods without any blades and thick midveins with narrow and irregular blades to leaves with long petioles and short terminal blades which curled upwards to form cup-shaped structures. A majority of the newly emerging leaves on these plants were abnormal, giving the plants the crab-leg-like appearance quite distinct from the appearance of the WT plants under the same conditions (FIG. 5A, panel 1). Similarly deformed leaves were seen less frequently on infected dsRDR6 plants kept at 27° C. (FIG. 5A, panels 3 and 4), very occasionally on infected WT plants, and never on mock-infected plants. Furthermore, plants kept at 27° C. were beginning to flower at 5 weeks after infection and although they had notably fewer flowers than WT plants, the dsRDR6 infected plants had a much higher proportion of deformed filamentous flowers (FIG. 5A, panel 5). Again, the increased proportions of leaf and flower abnormalities could not be directly attributed to differences in viral accumulation levels in plant tissues at these advanced stages of infection.

The leaf abnormalities described above closely resembled those documented for both Arabidopsis and N. sylvestris mutant plants with gain-of-function mutations in PHB, PHV and REV genes and for transgenic Arabidopsis as well as N. benthamiana plants expressing virus-encoded suppressors of RNA silencing. This suggested that the dsRDR6 plants, the silencing suppressor encoded by TMV, the small subunit of TMV replicase, might be interfering with the miRNA-guided developmental regulation, as exemplified by the miR165-mediated degradation of PHB, PHV and REV mRNAs. The accumulation levels of both the mRNA of NbPHV and miR165 in the apical tissues of the infected plants were evaluated. For this purpose, RNA was extracted exclusively from the terminal 15 mm of stems and branches of uninfected and infected WT and dsRDR6 plants held at 21 and 27° C.

The RNA samples were first subjected to hybridization with a TMV probe to confirm the presence of TMV-GFP RNA in the apical tissues. The infected dsRDR6 plants accumulated TMV-specified RNA to only slightly higher levels in the apical tissues than did WT plants at both temperature conditions (FIG. 5B, panel I). This, however, may not completely reflect the difference in apical invasion because the true apical meristems constituted only a small portion of the tissues we collected for RNA extraction. The TMV genomic RNA band migrates slightly more quickly in the 27° C. samples than in the 21° C. samples, resulting from more frequent deletion of the GFP insert at the higher temperature.

RNA blot hybridization was then carried out using an Nb-PHV probe. The results presented in FIG. 5B, panel II, show significant differences in the levels of NbPHV mRNA in these tissues. While the uninfected dsRDR6 plants expressed Nb-PHV at a lower level than the WT plants (FIG. 5B, panel II, lanes 3 and 4 versus lanes 1 and 2), this did not seem to visibly affect N. benthamiana development. However, the TMV-infected dsRDR6 plants accumulated NbPHV mRNA to substantially higher levels than did WT plants (FIG. 5B, panel II, lanes 7 and 8 versus lanes 5 and 6). Moreover, the increase in the level of NbPHV mRNA was most marked when the infected dsRDR6 plant samples were compared to their uninfected counterparts (FIG. 5B, lanes 7 and 8 versus lanes 3 and 4). The difference was less dramatic for plants kept at the higher temperature (FIG. 5B, lanes 11 and 12 versus lanes 9 and 10), consistent with the less striking leaf abnormalities observed with these plants.

To ascertain that the difference in the NbPHV mRNA levels was not caused by uneven loading of samples, the RNA samples were further subjected to hybridization with a probe that detects NbACT mRNA, which is not known to be targeted by miRNA. As shown in FIG. 5B, panel III, while minor variations in NbACT mRNA level are visible, they clearly do not account for the difference in the levels of NbPHV mRNA.

These results collectively illustrate that the increased accumulation of NbPHV MRNA in the apical tissues of TMV-GFP-infected dsRDR6 plants was highly correlated with the degree of abnormal leaf and floral development, further supporting the notion that the miRNA-mediated regulation of NbPHV expression is most likely interfered with inside the apical tissues of the infected dsRDR6 plants. The accumulation levels of miR165 were unchanged for both healthy and infected WT and dsRDR6 plants (FIG. 5B, panel IV), suggesting that the TMV invasion interrupted the miR165-mediated targeting of NbPHV mRNA rather than the miRNA production. This result is not unexpected given that viral silencing suppressors are known to act at similar steps on the miRNA-and siRNA-mediated pathways and given the finding that the silencing suppressor encoded by tomato mosaic virus, a virus closely related to TMV, blocks the utilization of siRNAs.

Together, these results indicate that the lack of NbRDR6-mediated defense permits TMV-GFP to invade the shoot apices more efficiently, allowing its silencing suppressor to interrupt the miRNA-mediated regulation of expression of these HD-ZIP III genes in leaf primordia. To determine whether TMV-GFP did indeed invade the shoot apices of dsRDR6 plants more readily, the infection experiments were repeated with TMV-GFP at 21° C. and the shoot apices of infected plants were inspected at 9 dpi, at which point systemic symptoms were evident. To ensure effective expression of GFP in the systemic leaves, these leaves were first checked for distribution of GFP fluorescence under long-wave UV light. For both dsRDR6 and WT, about half of the infected plants showed extensive vein-aligned networks of GFP fluorescence in the systemic leaves, suggesting that both types of plants supported systemic spread of TMV-GFP to similar levels. The uppermost shoot tissues, of about 2 mm in length, were detached from these plants and examined under a confocal laser scanning microscope (FIG. 5C). Usually, three developing flowers were visible on each shoot, with the first one (F1) being a floral primordium and the third (F3) possessing all floral parts but remaining unopened. GFP fluorescence was never seen in the apical tissues of any of the four infected WT plants examined (FIG. 5C, panels 3 and 4), but it was clearly visible in all four of the infected dsRDR6 plant apices, within at least one of the developing flowers. Panel 5 of FIG. 5C shows a floral primordium with GFP fluorescence visible over the entire structure, whereas panel 6 shows a more developed F3 stage flower with even more evident GFP fluorescence. These results strongly suggest that the apical tissues of dsRDR6 plants are indeed more susceptible to sustain TMV-GFP infection than those of WT plants, clearly implicating NbRDR6 in the antiviral defense system operating in the shoot apices.

What is claimed is:

1. A polynucleotide construct comprising an RNA dependant RNA polymerase 6 operably linked to a promoter sequence to allow expression of RNA dependant RNA polymerase 6 in plants.

2. The polynucleotide construct of claim 1, wherein the promoter is a constitutive promoter.

3. The polynucleotide construct of claim 2, wherein the promoter is a cauliflower mosaic 35S promoter.

4. The polynucleotide construct of claim 1, wherein the promoter is an inducible promoter.

5. The polynucleotide construct of claim 1, wherein the promoter is a tissue preferred promoter.

6. A plant transformed with a polynucleotide construct comprising an RNA dependant RNA polymerase 6 operably linked to a promoter sequence to allow expression of RNA dependant RNA polymerase 6 in plants.

7. The plant of claim 6, wherein the plant is selected from the group consisting of cereals, pulses, tubers and seed crops.

8. The plant of claim 6, wherein the plant is a soybean plant.

9. The plant of claim 6, wherein the plant is a maize plant.

10. The plant of claim 7, wherein the promoter is a constitutive promoter.

11. The plant of claim 10, wherein the promoter is a cauliflower mosaic 35S promoter.

12. The plant of claim 7, wherein the promoter is an inducible promoter.

13. The plant of claim 12, wherein the promoter is induced by a viral infection of the plant.

14. The plant of claim 7, wherein the promoter is a tissue preferred promoter.

15. A method of conferring on a plant resistance to a broad spectrum of plant viruses comprising the step of transforming the plant with a polynucleotide construct comprising an RNA dependant RNA polymerase 6 operably linked to a promoter sequence to allow expression of RNA dependant RNA polymerase 6 in the plant.

16. The method of claim 15, wherein the plant virus is of the genera Potexvirus.

17. The method of claim 15, wherein the plant virus is of the genera Carmovirus.

18. The method of claim 15, wherein the plant virus is of the genera Tobamovirus.

19. The method of claim 15, wherein the plant is selected from the group consisting of cereals, pulses, tubers and seed crops.

20. The method of claim 15, wherein the plant is a soybean plant.

21. The method of claim 15, wherein the plant is a maize plant.

22. The method of claim 15, wherein the promoter is a constitutive promoter.

23. The method of claim 22, wherein the promoter is a cauliflower mosaic 35S promoter.

24. The method of claim 15, wherein the promoter is an inducible promoter.

25. The plant of claim 24, wherein the promoter is induced by a viral infection of the plant.

26. The plant of claim 15, wherein the promoter is a tissue preferred promoter.

27. An expression vector comprising a construct comprising an RNA dependant RNA polymerase 6 operably linked to a promoter sequence to allow expression of RNA dependant RNA polymerase 6 in plants.

28. A plant host cell comprising a construct comprising an RNA dependant RNA polymerase 6 operably linked to a promoter sequence to allow expression of RNA dependant RNA polymerase 6 in the plant host cell.
